(19) United States
(12) Patent Application Publication
(10) Pub. No.: US 2017/0188932 A1
(43) Pub. Date: Jul. 6, 2017

(54) SYSTEM AND METHODS FOR NEUROLOGICAL MONITORING AND ASSISTED DIAGNOSIS

(71) Applicant: Brainscope Company, Inc., Bethesda, MD (US)

(72) Inventors: Michael E. SINGER, Bethesda, MD (US); Gregory M. MAHOWALD, Kensington, MD (US); Leslie S. PRICHEP, Mamaroneck, NY (US); Samanwoy GHOSH DASTIDAR, North Potomac, MD (US)

(73) Assignee: Brainscope Company, Inc.

(21) Appl. No.: 15/357,139

(22) Filed: Nov. 21, 2016

Related U.S. Application Data
(60) Provisional application No. 62/273,677, filed on Dec. 31, 2015.

(51) Int. Cl. A61B 5/00; A61B 5/0476; G09B 7/00

(52) U.S. Cl. CPC A61B 5/4064; G09B 7/00; A61B 5/0022; A61B 5/0476; A61B 5/742; A61B 5/002

(57) ABSTRACT

Methods and systems for assessing brain activity and collecting information related to a subject's condition and brain electrical activity are provided. The methods and systems include collecting and transferring data related to the neurological state of a subject. The systems and methods further include performing a first assessment of the collected data using an assessment device and performing a second assessment of the collected data using a computer. The methods further include comparing the first assessment and the second assessment and recording whether the first assessment and second assessment are consistent.

### CROSS-REFERENCE TO RELATED APPLICATIONS

This application claims priority from U.S. Provisional Patent Application No. 62/273,677 filed on Dec. 31, 2015, the entire disclosure of which is incorporated herein by reference.

### TECHNICAL FIELD

The present disclosure pertains to systems and methods for monitoring and evaluating subjects, and more specifically, to monitoring and evaluating neurological and neuropsyciatric conditions in a variety of clinical or field-deployed contexts using networked devices allowing configurable interaction, data delivery and data collection involving subjects and those attending to the neurological or neuropsychiatric care of subjects.

BACKGROUND

There are many medical and surgical situations that require prompt and accurate diagnosis or triage to ensure optimum outcomes. However, it is often difficult to get a subject to a hospital or other site that has the most up-to-date diagnostic resources, and/or has staff available for accurate and rapid subject assessment. Further, as new medical information is generated through the experience of health care professionals at different locations, there can be significant time lags before such information is disseminated to other professionals or incorporated into technology that helps implement diagnosis or therapy. One approach to updating information on a central location from a local device is described in commonly assigned U.S. Pat. No. 8,577,451, which is incorporated herein by reference. In addition, improved systems for documenting the neurologic or neuropsychiatric condition of a subject over time and using the documented information to guide subject evaluation and treatment are needed. In neurological assessments, different information to be collected or presented during the process. For example, head trauma assessments for athletes by sports medicine clinicians or volunteers may involve different protocols and procedures than battlefield assessments by military medics. Similarly, the information collected during different head and/or brain health assessment contexts will vary. As well, an assessment of a subject having no prior historical information regarding neurologic health might vary from one on a subject undergoing longer-term monitoring or involving comparisons to a subject's own prior or baseline information. The type of information presented or solicited that is appropriate at the time of an assessment may vary depending on a subject's context and history. One approach for providing information to a user regarding a disease state over time is described in the commonly assigned U.S. Pat. No. No. 8,579,812, which is incorporated herein by reference.

### SUMMARY

It is accordingly an object of the systems and methods of the present disclosure to improve field diagnosis of traumatic brain injury, including concussion. It is a further object of the present disclosure to improve networked technological environments to provide connected neurologic assessment tools for assessing subjects in varying contexts, and managing those networked devices. It is a further object of the systems and methods of the present disclosure to allow context-sensitive delivery of assessment interfaces and assessment tools and collecting information.

The present disclosure solves several technical problems applicable to telecommunications of information in various field-deployed medical evaluation environments and contexts. Discussions of problems or improvements described herein should not be construed as an admission that the problems discussed were recognized in the prior art. Various aspects of the present disclosure are not recognized in the prior art. While schemes for assessment of concussion are described in the prior art, as is the generic ability to network devices in a healthcare environment, none recognize the specific challenges of performing an urgent concussion assessment robustly and accurately, notwithstanding the status of the assessment tool's connectivity. Nor do they try to accommodate it, as do the various embodiments of the present disclosure. The overall performance of the assessment system is improved by the technical solutions proposed by various embodiments of the present disclosure, as discussed herein.

A networked system and method for monitoring or evaluating a neurologic state of a subject and for delivering and collecting information pertinent to a subject is provided. A first aspect of the present disclosure is a method for monitoring or evaluating a neurologic state of a subject, comprising collecting data related to the neurological state of a subject at a first location using a field-portable assessment device, transferring the data related to brain electrical activity to a computer located at a second location that is different from the first location, performing a first assessment of the collected data related to the neurological state of a subject using the field-portable assessment device, performing a second assessment of the transferred data related to the neurological state of a subject using the computer, comparing the first assessment and the second assessment, and recording whether the first assessment and second assessment are consistent.

A second aspect of the present disclosure is a system for monitoring or evaluating a neurologic state of one or more subjects, the system comprising a sensing device. The sensing device includes at least one electrode configured to detect a brain electrical signal, a first processor configured to convert subject input into data related to neurological state of a subject at a first location, and a communication system configured to transfer data related to neurological state of one or more subjects to a memory unit at a second location that is different from the first location. The first processor is further configured to provide a plurality of classifications of a neurological state of the subject based on the data related to neurological state.

A third aspect of the present disclosure is a method for network-based diagnostics in a neurologic assessment device, comprising determining whether a headset has been connected to an assessment device, acquiring a headset identifier from a headset, and determining authenticity of the headset using the headset identifier.

A fourth aspect of the present disclosure is another method for network-based diagnostics in a neurologic assessment device, comprising determining whether a headset has been connected to an assessment device, acquiring a headset identifier from a headset, transmitting the headset identifier to a remote location, and determining authenticity of the headset at the remote location using the headset identifier.

A fifth aspect of the present disclosure is method of maintaining a database of used electrode headsets used in a distributed group of neurological assessment devices, comprising acquiring one or more headset identifiers from an assessment device, and updating a databased using the headset identifiers using the headset identifiers acquired from the assessment device.

A sixth aspect of the present disclosure is another method of evaluating a neurologic state of a subject, comprising extracting features from the subjects brain electrical activity, neurocognitive assessments and/or clinical symptoms assessment, determining whether values of the extracted feature fall within or outside their normal ranges, wherein the normal ranges are defined by a reference database comprising brain electrical activity data, neurocognitive assessments data and clinical symptoms data collected from a control population group exhibiting normal brain function, calculating a composite score of normal brain function using normal and abnormal features, and generating an index of brain function or normality indicator using the indicator score, wherein the index or normality indicator expresses the neurologic state of the subject as a percentile relative to normal brain function exhibited by the control population group. The index of brain function can be conceptualized as an indicator of normality.

According to an aspect of the invention, an apparatus for monitoring for evaluating a neurologic state of a subject by a user is disclosed, comprising one or more electrodes disposable on the subject and configured to detect brain electrical signals of the subject; an assessment device having a processor and a memory, the assessment device configured to receive the brain electrical signals and the processor configured to perform a first assessment of the brain electrical signals; a computing device, separate from the assessment device, having a processor and a memory; and a first communications channel between the assessment device and the computing device, wherein brain electrical signals can be communicated from the assessment device to the computing device when the first communications channel is open; wherein the computing device processor performs a second assessment of the brain electrical signals.

Other embodiments and aspects of this disclosure are contained in the accompanying drawings, description, and claims. Thus, this summary is exemplary only, and is not to be considered restrictive.

### BRIEF DESCRIPTION OF THE DRAWINGS

The accompanying drawings, which are incorporated in and constitute a part of this specification, illustrate the disclosed embodiments and together with the description, serve to explain the principles of the various aspects of the disclosed embodiments. In the drawings:

FIG. 1 illustrates a system for evaluating a neurologic state of a subject, according to an illustrative embodiment of the present disclosure.

FIG. 2 illustrates a handheld assessment system and server, according to an illustrative embodiment of the present disclosure.

FIG. 3 illustrates a process for the assessment of a patient according to an illustrative embodiment of the present disclosure.

FIG. 4 illustrates a process for evaluating the state of an assessment system according to an illustrative embodiment of the present disclosure.

FIGS. 5A, 5B, and 5C depict user interface displays according to illustrative embodiments of the present disclosure.

It is to be understood that both the foregoing general description and the following detailed description are exemplary and explanatory only and are not restrictive of the disclosed embodiments, as claimed.

### DESCRIPTION OF EXEMPLARY EMBODIMENTS

Reference will now be made in detail to exemplary embodiments according to the present disclosure, examples of which are illustrated in the accompanying drawings. Wherever possible, the same reference numbers will be used throughout the drawings to refer to the same or like parts.

The present disclosure provides systems and methods for evaluating the neurological state of a subject and providing an assessment of a subject's neurological state. As used herein, the adjective “neurologic” or “neurological” when used to describe states and assessments shall be understood to apply to neurological and/or neuropsychiatric conditions. In some embodiments, the assessment involves brain electrical activity information obtained from a subject. Throughout the disclosure, the terms “brain electrical activity data” and “brain electrical information” is used interchangeably. In some embodiments, the assessment involves clinical assessments, based on questions or observations obtained from a subject, or from cognitive performance tests such as simple or procedural reaction time assessments, visual/spatial processing assessments, or short-term memory tasks. In some embodiments, the assessment can be based on information contained in a database including numerous brain electrical activity data sets, including, for example, features related to normal brain state and/or a variety of different diseases, pathologies or brain injury states. In some embodiments, the systems and methods of the present disclosure provide tools for assessing a subject's neurological state based on a database containing brain electrical activity data for numerous subjects, as well as methods and systems for verifying the assessment, updating the database to expand the data included therein, and/or improving the range of diseases or conditions that may be identified, and/or improving diagnostic sensitivity and/or specificity using the database.

In some embodiments, networked devices can be updated remotely. This advantageously allows onboard program code and/or parameters to be changed via remote commands over networks. In other illustrative embodiments, these updates can be applied institutionally with or without network support, so that all devices used by a group or institution can be updated with the same information.

In some exemplary embodiments, raw (unprocessed) data is transmitted to a central network, and the preprocessing and diagnostic processing of the data takes place on the server side in addition to on the local device. Advantageously, the approach then compares the results of the local device to the server-side results to ensure consistency.

In some embodiments, the systems and methods provide information about a subject to supplement brain electrical activity information, clinical assessments and cognitive tests provided by the local device. Such information in illustrative embodiments includes whether a subject is CT+ or CT− for structural brain injury, and can be entered on or otherwise acquired by the local device, or if such information is not available at the time of device use, via a web portal by the subject, or by clinicians or by the institution. The subject's profile maintained on a server database is updated to contain any additional information. In exemplary embodiments, these additional data can be used to update, optimize and/or enhance diagnostic and treatment algorithms.

In an illustrative embodiment, the system comprises at least one local apparatus configured to collect information related to a condition of a subject, and which contains a processor for calculating information related to a subject's condition. In illustrative embodiments, the information can include brain electrical information, cardiac rhythm information, clinical assessments, physiologic measurements, such as, blood pressure, blood oxygenation, skin conductance, blood biomarkers, eye tracking, etc., and/or cognitive functioning data. The illustrative networked system further can include at least one terminal for displaying, inputting and managing information related to a condition of a subject, the interface based on the identity of the user. The illustrative system also includes an analysis system. The analysis system, which in an illustrative embodiment is a computer or server in a network or on the cloud, comprises a reception system configured to receive data related to the subject's condition from the local apparatus and/or the terminal, and a processor configured to assess a condition of a subject and to identify a data set including information related to the subject's condition. The networked illustrative system also comprises a communication system configured to communicate the data set to a user.

A method for communicating health information is provided. The method comprises collecting information related to the condition of a subject and entering the information related to the condition of a subject in a computerized data collection system. The analysis system configured to process the information to assess a disease stage of the subject, identify a data set including information related to the subject's condition, and communicate the data set to a user. Based on the identity of the user and/or the content of the information, an illustrative analysis system adaptively constructs a user interface (UI) to display to the user on either a terminal, a local device, or any handheld device with web connectivity.

An illustrative system for delivering health information is provided. The system can comprise at least one reception device configured to receive information related to a condition of a subject; an analysis system including a receiver configured to receive data related to the subject's condition and a processor circuit configured to process data related to the subject's condition, and to identify a data set related to the subject; and a communication system configured to selectively communicate the data set based on a requester's identity.

As described further below, the systems and methods can include a sensor and processor at a first location to collect data related to a subject's brain electrical activity. The data can be transferred to a centralized database, which may be at a different location than the data collection site. The centralized database can be updated with data at multiple time points from multiple local devices and/or from multiple subjects to allow automatic generation of diagnostic and treatment algorithms. In addition, the data can be stored in a second database, which includes longitudinal data from a specific subject, thereby allowing continuous monitoring of that subject's neurological status and/or providing ongoing treatment guidance.

The systems and methods can also facilitate treatment planning and decision-making. In some embodiments, the systems and methods provide a database of treatments administered to the same subject over time (longitudinal data) or to a population of subjects along with the effects of the treatments on electrical activity and/or other assessments of neurological status. Evaluation of the progression of a subject's assessment over time advantageously provides for patient safety, for example in the case of concussion where a return of an athlete or a warfighter to action could result in re-concussion, with its attendant health risks.

In some embodiments, the systems and methods provide a treatment suggestion or recommendation system for subjects whose symptoms, brain electrical activity, and/or other neurological assessments most closely approximate those of other subjects previously stored in the database or based on established standards-of-care. The type and scope of the treatment suggestions can be tailored to the user. For example, if the subject is accessing suggestions via a terminal, the information will be tailored to the subject, displaying more informational and explanatory text. If a clinician is accessing suggestions using the local device during assessment of the subject, then the information may be more diagnostic and direct, without the need for explanatory text. The UI can also be configurable or adaptive over time, where information previously displayed to a user can be omitted in the future. If a certain period of time has elapsed since the user has last seen the information, the information can be redisplayed to ensure the user is current.

In illustrative embodiments, users can be identified by the networked system via login credentials. In some embodiments, the system can be used to distribute information related to a treatment recommendation, types of care, support provisions, financing of healthcare, health maintenance, prevention of complications (e.g. appropriate return to activity, duty, play, etc.), medication side effects, and/or any other health information that may be of time-specific interest to a subject or other persons involved in the care of a subject. In some embodiments, the health information can be communicated to the subject or others over the time course of a subject's disease. Further, in some embodiments, the system and methods of the present disclosure can facilitate monitoring of the progression or stabilization of a subject's disease. In some embodiments, the systems of the present disclosure will assist in distributing timely healthcare information to the subject as their disease or condition develops. In some embodiments, the systems will provide advance information about likely upcoming states or stages of a subject's condition, and information relevant to the next or following stages of the condition.

Access to timely medical information is important for patients, medical professionals, and others involved with the care and treatment of acute head trauma patients or those experiencing ongoing symptoms related to head injury. Coordinating delivery of appropriate information to assist in diagnosis, clinical management of treatments, lifestyle planning, to provide emotional support, or otherwise assist in coordinating subject and clinician decision-making and understanding can be difficult, and improved systems and methods for providing appropriate information and the appropriate time are needed.

Medical information systems, including medical databases and data networks have been developed primarily to assist in storing and communicating medical record data to assist in prescribing and controlling medical resources. In addition, remote patient monitoring systems have been developed that allow continuous or periodic monitoring and collection of patient health information, while facilitating communication between patients and health care professionals. However, there remains a need for systems centrally managing information collection and distribution among local assessment device and terminals. The local assessment device can be apparatuses and/or apparatuses implementing methods, as described in U.S. Pat. Nos. 7,720,530, 7,904,144, 8,364,254, 8,838,227, 8,948,860 and Published U.S. Patent Application No. 2011/0038515, each of which is incorporated herein by reference. The system of the present disclosure can employ a network or cloud-based system connecting the local devices when connectivity is available. Local devices are designed to be self-contained and provide brain access to health assessments with or without connectivity. Network connectivity permits assessing the time course of a subject's condition over time and providing timely information related to available diagnostics, recommended to possible treatments, lifestyle issues, finances, and numerous other issues affecting patients. Connectivity also permits wireless or wired transfer of acquired data from the local device to one or more central database(s). This information can be used not only in the assessment of the subject at a central location, but also for the construction of classifiers and devices utilizing them as described in the U.S. Pat. Nos. 8,792,974, 9,198,587 and Published U.S. Patent Application No. 2014/028172, each of which is incorporated herein by reference.

### Neurological Monitoring System

The systems and methods of the present disclosure may include one or more systems or devices for receiving information related to the condition of a subject. As described in more detail below, the systems or devices can include numerous different health information input systems and/or diagnostic apparatuses that are configured to receive information related to a disease or condition of one or more subjects. In some embodiments, these devices and systems can be designed to specifically collect information related to the condition of a patient in order to assess the time status of a subject's condition. In other embodiments, these devices or apparatuses can be designed for other specific or general healthcare or health monitoring purposes, but can include components that allow recording and/or transfer of information related to the subject's condition or disease. Further, in some embodiments, as described in more detail below, the systems and methods of the present disclosure can employ multiple apparatuses or devices for collecting healthcare information and can be distributed as a network of devices that are configured to receive information related to a patient's condition or disease.

As used herein, “brain electrical activity” will be understood to refer to any measurable electrical activity from the central nervous system, including electrical activity detected by any means, including for example, electroencephalography, and/or brainstem or other auditory, visual, or other sensory/somatosensory evoked responses, or cognitive evoked potentials. The term “subject” is used to refer to the individual who is assessed (e.g., a patient, an athlete, a warfighter, a study participant), and “user” is the operator of a local assessment device or a terminal. A “terminal” is an input/output device or information display of any kind. The terminal user can be the subject. It is possible that the user of the local device can be the subject, although the user of the local device is usually not the subject when brain electrical activity data is being collected. In addition, the local device can be a terminal. Following collection of brain electrical information, the user of the local device can be the subject during clinical assessments and cognitive testing. As discussed herein, connectivity between local devices and a central “server” may be a central server in a fixed location, in the “cloud,” installed or located at an institutional site such as a hospital or sports arena, or a combination of the above. “Connections” and “connectivity” discussed herein can include internet connections, telephone connections, direct wire links, wireless and other suitable connection types. In addition, different types of data connections can be used for different types of devices and analysis systems. For example, some patient monitoring systems may include wireless data connections that are configured to monitor patient physiologic conditions directly. In addition, some patient data information input systems can include static connections, as for example, connections at a doctor's office or other healthcare facility, and such static connections may be configured to transmit information over a landline or telephone connection.

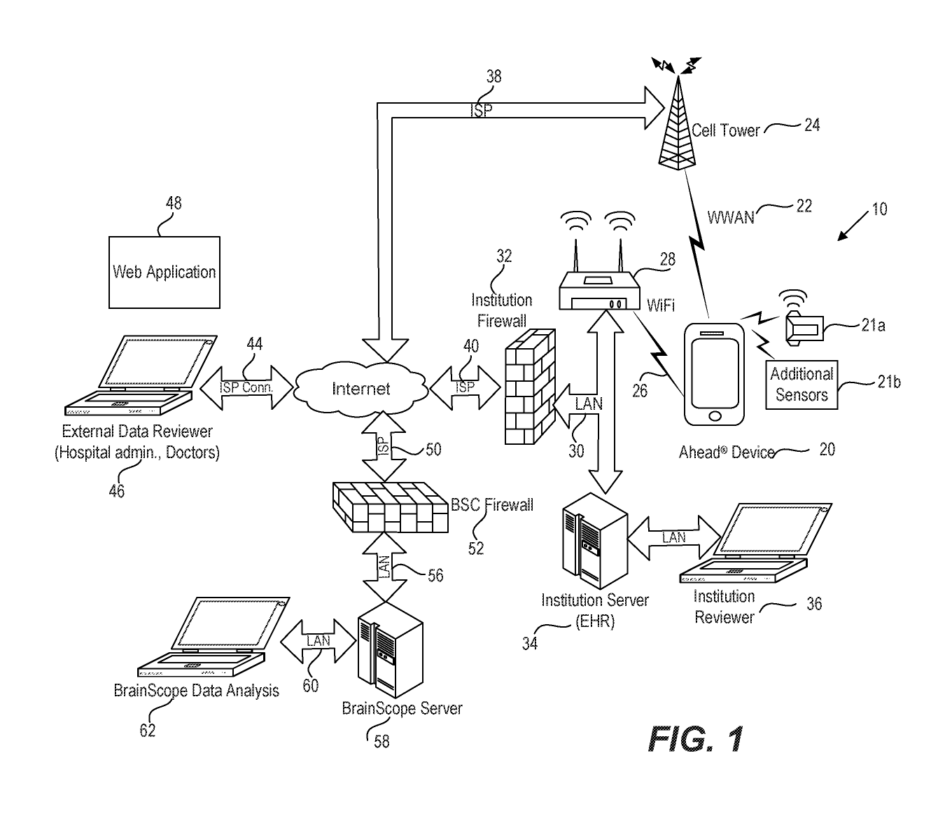

FIG. 1 illustrates a healthcare system 10 for managing information related to the neurologic state of a subject, according to certain embodiments of the present disclosure. In some embodiments, the system includes an analysis system 20 in the form of a local assessment device for evaluating a neurologic state of a subject. In the illustrative embodiments, the analysis system 20 is a handheld device, which can be implemented on a smart phone or other handheld computer. The system can include a sensing device 21a including at least one electrode configured to detect a neurologic electrical signal. Additional sensing devices 21b may be connected to analysis system 20 to collect one or more biological signals, perform and/or collect other clinical or neurophysiological measurements and/or results, or perform clinical symptoms assessments, for example an ocular function test as described in commonly assigned U.S. patent application Ser. No. 15/339,640. The sensing devices may be connected to the handheld device via wired or wireless connection (for example, using Bluetooth (BT), Bluetooth Low Energy (BTLE), etc.). The system 20 can further include a processor configured to convert the electrical signal into data related to brain electrical activity of at least one subject at a first location. The analysis system 20 can be connectable via wireless WAN link 22 to a cellular network 24. A local wireless link 26 to a Wi-Fi router or node 28 or other network connectivity node. The node 28 in an illustrative embodiment can be part of a local area network (LAN) 30. LAN 30 in an illustrative embodiment is placed behind a firewall 32, and can contain an institutional server 34 which is connected to one or more terminals 36. Terminals 36 permit review and management of data collected by any analysis systems 10 on the network, as well as subject data records resident on the institutional server 34. For a hospital or healthcare institution, the data records can comprise an electronic health record (EHR), or for sports team or military units, the institutional server can contain records related to individual athlete as well as the collective.

In some embodiments, analysis system 20 may be configured to include a display for displaying visual target viewable by a subject. Further, an illustrative embodiment of analysis system 20 can include a distance measurement component that measures a distance from analysis system 20 to a subject. Analysis system 20 can further include a display for displaying, for example, instructions, error messages concerning the operation of analysis system 20, feedback information to a user, etc. In an illustrative embodiment a timer can be included in analysis system 20, having a display for displaying a time of an ocular function test, for example.

In some embodiments, the distance measurement component of analysis system 20 may include a sensor that enables analysis system 20 to measure a distance from analysis system 20 to a subject, for example the subject's face, when a button of analysis system 20 is pressed by the user or a clinician in response to specific observations as part of each test. Distances can also be continuously measured.

In some embodiments, the distance measurement component may be configured to include the use of IR (infrared) or optical means to perform the distance measurement. In some embodiments, the distance measurement component may be configured to include an array of infrared sensor transmitter/receiver pairs to improve distance measurements (e.g., accuracy). A single infrared sensor transmitter/receiver pair may have a narrow optical field of view that typically range from about 10 degrees to about 15 degrees. This requires an accurate positioning and alignment of analysis system 20 and reduces measurement accuracy at longer distances. Utilizing an array of infrared sensor transmitter/receiver pairs may effectively increase the optical field of view by, for example, 2-fold from a range of about 10 degrees to about 15 degrees to a range of about 20 degrees to about 30 degrees, respectively. The array of infrared sensor transmitter/receiver pairs may be mechanically spaced to enable an overlap in the optical field of view. Accordingly, the sensitivity of alignment of assessment device 30 may be minimized and the distance measurement accuracy at longer distances may be improved.

Additionally, the array of infrared sensor transmitter/receiver pairs may provide a measurement of azimuth and elevation of analysis system 20 and a positional feedback. Further, the positional feedback may be incorporated into a display of analysis system 20 to provide a visual indicator. The visual indicator may include a signal strength indicator that changes color as analysis system 20 becomes optimally positioned, or a bullseye type of target that displays and moved toward the center as assessment device becomes optimally positioned. Further, the positional feedback may be used to generate an audible tone to guide the user in optimizing the position and alignment of analysis system 20 relative to the target. Thus in some embodiments, by employing an array of infrared sensor transmitter/receiver pairs, distance measurement inaccuracies may be addressed. Further, a user may be assisted in positioning and/or aiming analysis system 20 to minimize the sensitivity associated with positioning analysis system 20 correctly.

Internet service provider (ISP) link 38 is accessible via the cellular network 24, and ISP link 40 is accessible behind firewall 32, providing access to the internet 42 and the cloud. The internet can connect various access nodes used by the system, and also to cloud-based resources (e.g., networks, servers, storage, applications and services) that are accessible substantially instantaneously.

An ISP link 50 provides internet connectivity behind firewall 52 to central LAN 56 connecting central server 58. Central server 58 is connectable to LAN 60 to one or more terminals 62. The central server can house a software image for all local devices in the field to access and upgrade. The installed base of local devices can be monitored centrally to allow trending of data across the installed base. Further, the central server can identify device anomalies, improvements, etc. as further discussed below with reference to certain illustrative embodiments.

A remote connection into the internet 42 via ISP link 44 with one or more terminals 46 allows interaction with the neurological monitoring system, in illustrative embodiments via a web application 48. Using the appropriate credentials, an external user can receive data from and transmit data to the various parts of the system 10.

Data collected by the central server 58 can be used for algorithm development, as described in Published U.S. Patent Application Nos. 2011/0038515 and 2014/028172, and U.S. Pat. Nos. 8,792,974 and 9,198,587, which are incorporated herein by reference. As further disclosed herein, data collected by various local devices 20 can be analyzed using data science techniques to identify correlations and patterns in data for future exploitation and research. Information can also be entered at a later point in time to confirm the truth of the assessment results performed by a local device, as discussed further below.

The central server also stores the results of previous assessments performed in the field. This warehoused data can be retrospectively evaluated by new or updated assessment algorithms to evaluate any changed outcomes, or other statistics.

In some embodiments, the analysis system 20 may be configured to analyze the data related to brain electrical activity and to provide an assessment of a neurological state of the subject based on the electrical activity. In other embodiments, the institutional server 34 or central server 58 can perform the analysis of the brain electrical activity or clinical assessments (as described below) in addition to or in place of the analysis performed by analysis system 20. Other servers or processors in the system 10 or on the internet 42 can also perform the analysis. The algorithms executed to evaluate the electrical activity can be executed in redundant locations to confirm the results obtained from the local device, or if the local device is detected to no longer be online on a first communications channel, then the results of the evaluation can be delivered via a second communications channel, that is to say another form of communication associated to the user, for example an email address, a text message or posting to a web-based resource.

The local device can include data entry facilities able to receive information related to the neurological state of a subject that is based on an evaluation technique other than the classification based on brain electrical activity data. In some embodiments, the data entry facilities are configured to receive information related to an evaluation technique not including evaluation of brain electrical activity data, such as electrocardiogram (ECG), clinical assessments, physiological measurements, or cognitive performance assessments.

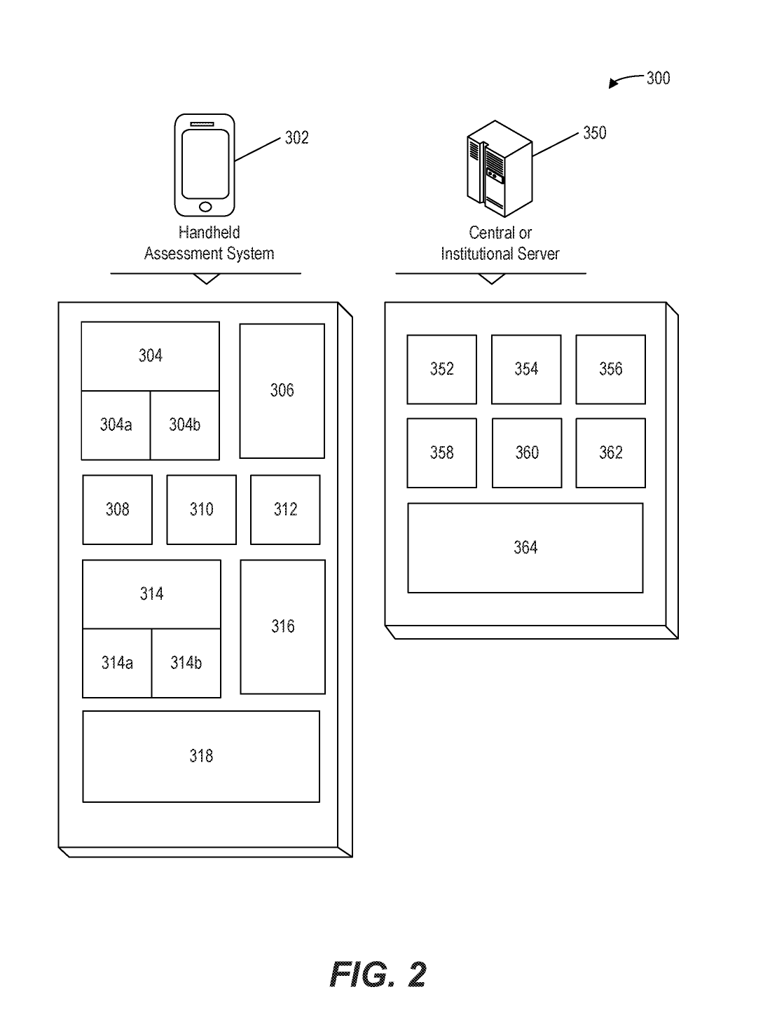

Turning to FIG. 2, an exemplary embodiment of the communications architecture 300 is illustrated. Handheld 302 in an illustrative embodiment can correspond to the local assessment device 20 as described above. The handheld 302 has various components to enable data communications. In the illustrative embodiment depicted in FIG. 2 these components can include those described herein. Network service discovery module 304 checks for available wireless or wired connectivity on any available mode, for example Wi-Fi at 304a, cellular at 304b, radio or other wireless band, or a wired connection. Network service discover module 304 can be configured to scan for available Wi-Fi access points, and can also attempt to authenticate and connect to identified networks. Software Upgrade Manager 306 communicates with a networked resource, such as central server 58 described above, to determine if a software upgrade package is available for download. The software upgrade manager 56 can be configured to perform retries, and also can verify the integrity, for example by performing a checksum operation, and/or authenticity of a downloaded package, for example by an encrypted package authentication protocol. In an exemplary embodiment, an MD5 checksum algorithm is employed, but other checksum schemes are also usable. Authentication can be achieved using a keyed-hash message authentication code (HMAC) such as SHA-1, but other schemes are also usable. The software upgrade manager 306 can, in exemplary embodiments, initiate a software recovery mode by performing a system reset, auto-recovery boot, or other processes for system management. Data backup manager 308 performs periodic uploads of some or all device data to a server, which in some illustrative embodiments is one or both the institutional server 34 and/or the central server 58. The data backup manager 308 can also be configured in some illustrative embodiments to perform a backup of some or all device data to another backup resource, such as a connected memory device or a networked resource. Email client 310 allows the handheld to transmit data using email protocols. For instance, in illustrative embodiments, session data and/or summary reports and/or links to access data via a terminal can be transmitted. In some embodiments e-mail protocols are employed for redundant communication or failure mode communications of assessment data or messages regarding device status. In an illustrative embodiment, the device can be configured to transmit an email when battery levels are critically low, or to send notices when bandwidth availability is too narrow to accommodate expected data transfer actions, such as the transmission of a DICOM file or complete EEG data file. Wi-Fi authentication and connection module 312 performs user authentication to grant permission to connect to a Wi-Fi access point. Authentication between a local device and central server shall be performed. HTTPS/FTP client services module 314 performs user authentication to grant permission to connect to an HTTPS/FTP resource, which in illustrative embodiments is secure 314a. HTTPS/FTP services can include file upload and download 314b.

The illustrative connectivity architecture 300 can also include modules for internet transactions over the World Wide Web. In exemplary embodiments, the architecture 300 can employ a coordinated set of constraints in the design of its components in a distributed hypermedia system. These components can be used to retrieve web pages and to send data to remote servers. In illustrative embodiments of interfaces with external systems, Uniform Resource Identifiers (URIs) can be employed. In an illustrative embodiment, representational state transfer architecture is employed. In an exemplary embodiment, information can be exchanged with handheld 302 using a Java Script Object Notation (JSON) parser, which translates human-readable Java text used to transmit data objects, for example attribute—value pairs. In an illustrative embodiment, XML can be employed. Transactions using these protocols can be managed by client/parser module 316.

The handheld 302 can also include a bootloader and auto-recovery module 318, which can advantageously be configured to perform device upgrades via an auto-recovery mode without user interaction.

Architecture 300 can include a server 350, which in an illustrative embodiment can correspond to the central server 58 as described above, to the institutional server 34, or to another server. The server 350 has various components to enable data communications. In the illustrative embodiment depicted in FIG. 2 these components can include those described herein. HTTPS/FTP Server 352, which is secure in exemplary embodiments, allows the handheld 302 to upload, for example device configuration files, log files, patient data, summary reports and other files. HTTPS/ FTP server 352 also allows the handheld 302 to download files, such as software upgrades and other secure data, such as group subject files for storage on the handheld 302. HTTPS Server 354, which in exemplary embodiments can be secure, additionally provides web services to allow interaction with and display of patient data, as well as other interactivity, using the handheld 302 or other terminals as discussed above. Users manager and authorization module 356 in an illustrative embodiment facilitates the creation, management and authentication of users and devices granted permission to login to the server 350.

As discussed above, the illustrative connectivity architecture 300 can also include modules for internet transactions over the World Wide Web. In exemplary embodiments, the architecture 300 can employ a coordinated set of constraints in the design of its components in a distributed hypermedia system. These components can be used to retrieve web pages and to send data to server 350. In an illustrative embodiment, representational state transfer architecture is employed. In an exemplary embodiment, information can be exchanged with server 350 using a Java Script Object Notation (JSON) parser, XML or other standard. Transactions using these protocols can be managed by server/parser module 358. A cloud services module 360 can also be employed in exemplary embodiments to manage access to cloud-based online services, for uploading subject data or storing longitudinal information, timelines or other analytics for a subject or group of subjects using the same modules 352 and 354 used in communication with the local device 302. In illustrative embodiments, database 362 can store and track handheld parameters, configuration and log files, patient data and upgrade files, as well as other data. The database can also contain data used to determine whether a connected headset is authentic, which can take the form of permissible headset identifiers or algorithms for validating a headset using a headset identifier, for example using hash codes or other encryption techniques.

### Neurological State of the Subject

In many modern environments, a subject has an electronic record containing information about that subject. In a medical context, an EHR can be constructed to conform to ASTM E1384-07(2013) “Standard Practice for Content and Structure of the Electronic Health Record (EHR),” which is incorporated herein by reference.

In illustrative embodiments, the information related to the neurological state of a subject can take the form of brain electrical information (e.g., EEG) acquired using local device 20. One or more electrodes can be used to acquire electrical signals from a subject. In some embodiments, evoked potentials may also be facilitated using a stimulus emitter to elicit evoked potentials. Brain electrical signals, which may include spontaneous or evoked potentials are acquired from headset electrodes are passed to a processor in the local device, which executes instructions contained in memory for processing the acquired signals. As described further below, the signal in an illustrative embodiment are transmitted to servers for processing either instead of or together with the local device 20. In an embodiment consistent with the present disclosure, the signals are processed to remove noise, processed to extract features, and processed to classify the extracted features. At its core, brain electrical information such as EEG reflects functional brain injury and can describe features relative to expected normal values.

In illustrative embodiments, the information related to the neurological state of a subject can take the form of medical imaging data, which in an illustrative embodiment includes information in a DICOM (Digital Imaging and Communications in Medicine) compliant format. DICOM is a standard developed by the National Electrical Manufacturers' Association (NEMA) and is incorporated herein by reference in its entirety. Medical imaging data can also be in other formats, like HL7, JPEG or TIFF. Imaging data can include, in exemplary embodiments, data obtained from techniques such as X-rays, magnetic resonance imaging (MRI), computed tomography (CT), positron emission tomography (PET), magnetoencephalography (MEG), functional MRI (fMRI), near infrared spectroscopic imaging (NIRSI) and/or single-photon emission computed tomography (SPECT).

In illustrative embodiments, the information related to the neurological state of a subject can take the form of clinical assessments. For example, for assessing concussion, the clinical assessments can include a cognitive screening tool, such as the Standard Assessment of Concussion (SAC) from the Brain Injury Association, the Sports Concussion Assessment Tool—3 (SCAT3) from the Concussion in Sport Group, the Military Acute Concussion Evaluation (MACE) from the Defense and Veterans Brain Injury Center and the Diagnosis/Assessment of mTBI from Ontario Neurotrauma Foundation (OMF). The OMF guideline recommendations include modules including the Westmead Post-Traumatic Amnesia Scale (A-WPTAS), the Rivermead Post Concussion Symptoms Questionnaire, which can be used separately or together with the OMF assessment. Other neurological and neuropsyciatric conditions, e.g., post-traumatic stress disorder (PTSD), Chronic Traumatic Encephalopathy (CTE), etc., may be assessed using checklists or questionnaire that may be stored in local assessment apparatus 20. For example, checklists/questionnaire such as Primary Care PTSD Screen (PC-PTSD), Trauma Screening Questionnaire (TSQ), Startle, Physiological arousal, Anger, and Numbness (SPAN) scale, etc. may be used to assess post-traumatic stress disorders. In some embodiments, the neurocognitive assessment may be performed using a dynamic questionnaire created from expert neuropsychological assessment practices. The questionnaire may be designed to dynamically adapt to responses given by a subject, i.e., each subject may not be asked exactly the same set of questions. The accuracy of neurocognitive assessment is enhanced by tailoring successive clinical inputs based on responses or values of preceding steps.

In illustrative embodiments, other brief neuropsychological test batteries that assess attention and memory function, or other neuropsychological symptoms, can be used. In exemplary embodiments, these tools can advantageously be scored through tabulation, but also advantageously provide standardized questions that can be assessed individually for statistical correlations to other data from both the individual subject, but also across a broader population.

In illustrative embodiments, the information related to the neurological state of a subject can take the form of evaluations of eye saccades, disruption of ocular motility, strabismus or other eye movements or eye tracking. These movements can be tracked electronically, using cameras or other sensors, or using reading tests requiring eye movement that can be scored. In some embodiments, a camera located on the local assessment apparatus 20 can be used in performing all or part of the ocular evaluation. In some illustrative embodiments, the information related to the neurological state of a subject can take the form of an analysis of convergence insufficiency. Convergence insufficiency can be determined by an evaluation of eye alignment both at distance and at near, and the ability of the patient to converge as a focal object is slowly moved towards the eyes. Several tests can be employed, including the cover test, the near point of convergence test and the convergence amplitude test. In illustrative embodiments, evaluation of near point of convergence and/or near point of accommodation can be evaluated. Similarly, eye-tracking testing techniques may include paper-and-pencil-based forms, including for example vestibular-ocular motor screening (VOMS), saccades test cards, etc.

In some embodiments, the information related to the neurological state of a subject includes a number-naming or character-naming test. Such a test in exemplary embodiments evaluates the tracking ability of the eyes. For example, a test can be based on measurement of the speed of rapid number-naming of character-naming by having a subject read aloud a series of single-digit numbers of characters while scanning test cards of other indicia-bearing carrier or surface, which can be hard-copy or any other type of display. Standardized instructions can be used, which can include a prescribed time period. The exemplary test can include, for example, on practice (demonstration) card and three test cards. To perform the exemplary test, participates can be asked to read characters from left to right as quickly as possible but without making any errors. The time to compete the test and the number of errors can be recorded. A subject may read a row of numbers or characters displayed on an assessment device, for example assessment device 20 of FIG. 1 or an associated device 21b of FIG. 1. Timer of assessment device 20 may be used to record a time for scoring, and acts as a start/stop for stopwatch. A display of assessment device 20 may be used to denote an error message (e.g., how many numbers are read incorrectly) during the reading. Such time and error messages may be streamed to transmitted to server 58 running a software application thereon that supports an exemplary test routine, such as a reading or scanning test, for analysis, display and/or storage.

In illustrative embodiments, the information related to the neurological state of a subject can take the form of postural or balance assessments. In an exemplary embodiment, the Balance Error Scoring System (BESS) is used to provide an objective method of assessing static postural stability. The balance-testing regime consists three stances on two different surfaces: a double leg stance, a single leg stance and tandem stance; the two different surfaces include both a firm ground and a foam surface. In an illustrative embodiment, a subject's stances are scored to reveal possible indication of concussion.

In illustrative embodiments, the information related to the neurological state of a subject can take the form of timed cognitive performance assessments in order to measure attention, permanent and working (short-term) memory, simple or procedural reaction time, visio-spatial processing and the speed of a subject for processing mental tasks. Memory tasks such as memory of words and language, designs including patterns and word or symbol placement or substitution, colors or sequences of numbers, letters, etc. can all be used in illustrative embodiments. Illustrative examples of short-term memory tasks include match-to-sample and digit symbol substitution tests. In an exemplary embodiment, one or more timed tests are administered to a subject, and a score is calculated. In some embodiments, the score can be compared to a normative database to arrive at a scaled score. In other illustrative embodiments, the score is compared to the subject's own baseline score stored in memory either on the local assessment device 20 or on one or more servers.

In illustrative embodiments, the information related to the neurological state of a subject can take the form of ECG data. In an illustrative embodiment, an ECG device includes an electrode assembly configured to sense heart-related signals upon contact with a user's skin, and to convert the sensed heart-related signals to an ECG electric signal. ECG measurements and variations can be used in the assessment of neurologic health and autonomic reflex testing, which measures automatic changes in heart rate, blood pressure, or heart rate variability can help assess traumatic brain injury and concussion and their potential after-effects.

In illustrative embodiments, the information related to the neurological state of a subject can take the form of a non-invasive measurement of intracranial pressure. In an illustrative embodiment, Doppler ultrasound waves are transmitted into the intracranial and extracranial segments of the ophthalmic artery. In a further illustrative embodiment, the displacement of the tympanic membrane can be measured to assess intracranial pressure. In a further illustrative embodiment, EEG signals can be assessed to indicate intracranial pressure as described in mutually-assigned Published U.S. Patent Application No. 2014/0171820, incorporated herein by reference in its entirety. In a further illustrative embodiment, intracranial pressure can be calculated by evaluating the attenuation of a transcranial acoustic signal, for further example using an acoustic signal generator inserted into the ear.

The neurological state of the subject based on clinical assessments can be input into the local device 20 at the time of assessment, or entered at an institutional terminal 36.

In exemplary embodiments, the processor in analysis system 20 may apply one or more multimodal classifiers to combine the results/outputs from a plurality of assessment technologies (e.g., brain electrical activity data and results of neurocognitive and clinical symptoms assessments) and provide a multi-dimensional evaluation of the subject's condition. The development and application of a multimodal classifier is described in U.S. Pat. No. 8,792,974 and U.S. Patent Publication No. 2014/0289172, which are incorporated herein in their entirely. In one such embodiment, the processor in analysis system 20 is configured to extract quantitative features from the brain electrical activity and results of the physiological and neurocognitive assessments, and apply one or more discriminant or regression functions to classify an unknown subject as belonging to one of two or more neurological categories. In some embodiments, only quantitative features from a subject's brain electrical activity are used with a classifier to determine a subject's neurological state. In other embodiments, only quantitative features from results of the physiological and neurocognitive assessments are used with a classifier to determine a subject's neurological state.

Any type of linear or non-linear binary classifier (for example, Linear (or a higher order) Discriminant, Gaussian Mixture Model, logistic regression, etc.) may be used to classify a subject's brain state into one of two diagnostic categories. In some embodiments, a classifier may be used to classify a subject's brain state into one of several diagnostic categories. The classifier may be a single modality classifier, i.e., it uses features from a single assessment (e.g., brain electrical activity recordings), or the classifier may be multimodal, i.e., it uses features from two or more assessments (e.g., brain electrical activity recordings and clinical assessments). Use of a classifier to assess traumatic brain injury is described in U.S. Pat. No. 8,478,394 (“the '394 patent”), which is incorporated herein in its entirety. The '394 patent describes a method of building a single modality classifier and a system for classifying a subject's brain state into one of four categories indicative of the presence and severity of traumatic brain injury. As described in the '394 patent, three binary classifiers are used to classify a subject into one of four categories indicative of the presence and severity of traumatic brain injury. The four categories include: 1) abnormal brain electrical activity consistent with structural brain injury, 2) abnormal brain electrical activity consistent with non-structural injury with severe clinical manifestations of functional injury, 3) abnormal brain electrical activity consistent with non-structural injury with less severe manifestations of functional injury, and 4) normal brain electrical activity. One or more multimodal classifiers may also be used to classify a subject's brain state into diagnostic categories indicative of the presence and severity of traumatic brain injury.

In exemplary embodiments, instead of classifying a subject's brain state into a diagnostic category using a binary classifier (e.g., normal vs. abnormal, or disease present vs. absent), a determination is made of whether a subject's brain state is in the expected normal range or not. In such embodiments, a continuous indicator of normality, such as a score of brain function, is computed based on one or more feature scores acquired from a subject. The feature scores may be raw scores or they can be transformed into another mathematical score (e.g., z-score). A composite score, referred to herein as a “summary index score” may be calculated using the raw or transformed feature scores. In exemplary embodiments, one or more features (e.g., brain electrical activity features) may be converted to feature “z-scores,” which provide a statistical indication (in standard deviation units) of whether the underlying quantitative measure falls within a normal range. The normal range may be defined by an available normal control population. An “abnormal” feature may be identified by applying a z-score threshold which corresponds to a selected population percentile. In other words, the normal range of z-scores (standard deviation units) can be defined for example as |z|<=1.96 which will capture approximately 95% of the normal subjects, or as |z|<=1.28 which will capture approximately 80% of them.

The feature scores may be combined in many different ways to generate the summary index score. In one embodiment, a count of the abnormal features may provide the summary index score. The count may be expressed as a fraction of the feature pool considered for the particular computation. In another embodiment, a simple sum or a weighted sum of the abnormal features may provide the summary index score. In yet another embodiment, the summary index score may be computed using a weighted sum with additional weighting applied only to the abnormal features. A control group of abnormal population may be used to test whether the indicator score is on average “more abnormal” in those groups than in the control group of normal population and to test whether a significant gradation with severity is observed.

The set of features under consideration may be constructed to best reflect the neurological condition (e.g., concussion or TBI) that is being examined. For example, in the case of concussion, the features may be a set of EEG signal complexity features and/or EEG brain connectivity features(e.g., coherence, phase synchrony), and/or features reflecting changes in the frequency spectra. In illustrative embodiments, EEG complexity features can include one or more of compressibility, entropy (e.g., Shannon, Boltzmann-Gibbs, Tsallis, FuzzyEn, ApEn, SampEn, FApEn, FSampEn, etc.), multiscale entropy (MSE), fractal dimension, adaptability, scale-free measures or changes thereof over time. In illustrative embodiments, EEG brain connectivity features can include one or more of features disclosed in Thatcher, Robert W. et al., “EEG and Brain Connectivity: A Tutorial,” available at http://www.appliedneuroscience.com/Brain%20Connectivity-A%20Tutorial.pdf, incorporated herein by reference. Changes of these measures over time can also be used as features in illustrative embodiments. In illustrative embodiments, features reflecting changes in the frequency spectra can be focused on a particular band or bands, e.g. the alpha waves, and may further consider their change over time.

In some embodiments, the summary index score may be subjected to an additional mathematical transform (e.g., conversion to a percentile scale, or the mean/standard deviation of the summary score relative to a normal population). Either the summary index score or the transformed score may serve as the index of brain function. For example, in some embodiments, the score may be converted into a percentile scale through a non-linear statistical mapping. In such embodiments, a percentile Look-up Table (LUT) may be generated based on the index score calculated for a large database of healthy (normal) individuals. Based upon the index score distribution of this large dataset, the thresholds for each percentile are computed. The summary index score for a subject is then compared to this LUT to determine the assessment percentile. In exemplary embodiments, the percentile scale ranges from 0 to 100. The percentile indicates where a subject's score falls relative to the percentiles derived from the normal population, i.e., where the subject's score falls within the expected normal range. For example, a subject who gets a score x on the percentile scale (from 0 to 100) has a score below that of 100-x% of the “normal subjects” (where “normal subject” is defined by the control group of normally functioning population). The lower the percentile, the more the number of normal subjects that fall above the score of the subject. For example, for subjects with scores at the 10^{th} percentile, 90% of the normal population has scores above that of the subject. Thus, the percentile scale serves as an intuitive index which is interpreted relative to a population of normal subjects.

In exemplary embodiments, a subject's summary index score or the transform (e.g., the percentile score) may be compared to several subject groups within a diagnostic category (for example, subsets of TBI subjects) to determine whether the summary index score increases (or the percentile decreases) with functional severity and/or complexity of the disease/neurological condition. For example, an increase in the summary index score, or decrease in the percentile, may be observed in the following groups of concussion subjects (in order of severity): the mildly concussed subjects, moderately concussed subjects, structurally injured subjects with no measurable blood (as per CT scan read), structurally injured subjects with measurable blood (>=1 cc).

In exemplary embodiments, the index of brain function may be used to assess functional brain injury. For example, in some embodiments, the index may be used to assess functional changes in the brain as a result of concussion/mild TBI. In such an embodiment, the plurality of measures used to assess functional brain injury may comprise brain electrical features derived from EEG, since concussion/mild TBI is known to cause changes in brain electrical activity, e.g., measures of connectivity (i.e., changes in relationships between brain regions).

In some embodiments, a binary classifier may be used to first determine whether a subject has any structural brain injuries following a closed-head injury. Following the structural injury assessment, functional injury assessment may be performed by generating an index of brain function.

### Baselining

In any of the above assessments, as well as for brain electrical information, a baseline can be stored on the local device or on the institutional or central servers for a subject as part of the subject's profile. Comparisons of current assessment results with baseline values can be used in illustrative embodiments for identifying neurological issues.

A patient's baseline information can, in illustrative embodiments, include basic demographic information and descriptive information such as age, height, weight, occupation or team position, as well as relevant health history such as previous concussions, learning disabilities, preexisting speech pathologies, medications, etc.

A subject's brain electrical activity under normal conditions, i.e., prior to injury or known advent of a disease condition, can be collected and stored as part of the subject's baseline. Also, in some embodiments, clinical results collected from one or more neurocognitive and/or clinical symptoms assessments performed on a subject under normal conditions can be stored as part of the subject's baseline. The brain electrical activity data as well as the clinical results data collected pre-injury or pre-disease can be deemed as the subject's self-norm. Quantitative brain electrical activity features and clinical features acquired from the subject's self-norm can be compared to features acquired from the subject's brain electrical activity and clinical results following an injury or diagnosis of a disease condition. If a statistically significant or medically relevant change from the subject's self-norm is determined, a neurological indication may be produced showing the change and the ascribed medical significance of the change. The subject's brain electrical activity recording and clinical assessments may be performed continuously or periodically over a period of time and the acquired brain electrical features and clinical results features may be compared against the self-norm to determine change in the subject's brain state over time, or to track normal development or aging.

In exemplary embodiments, a baseline brain function index may be calculated and then subsequent computations of the index may be performed at intervals over a period of time. The subsequent index values may be compared to the baseline value to track the brain state of a subject relative to where the subject was when the baseline index was computed.

In some implementations, brain electrical activity features and/or clinical results features acquired from the subject's baseline brain electrical activity may also be used in the classification of the subject's neurological condition, for example, structural injury classification, or in the generation of an index of brain function for assessment of functional brain injury.

### Networked Assessment Processing

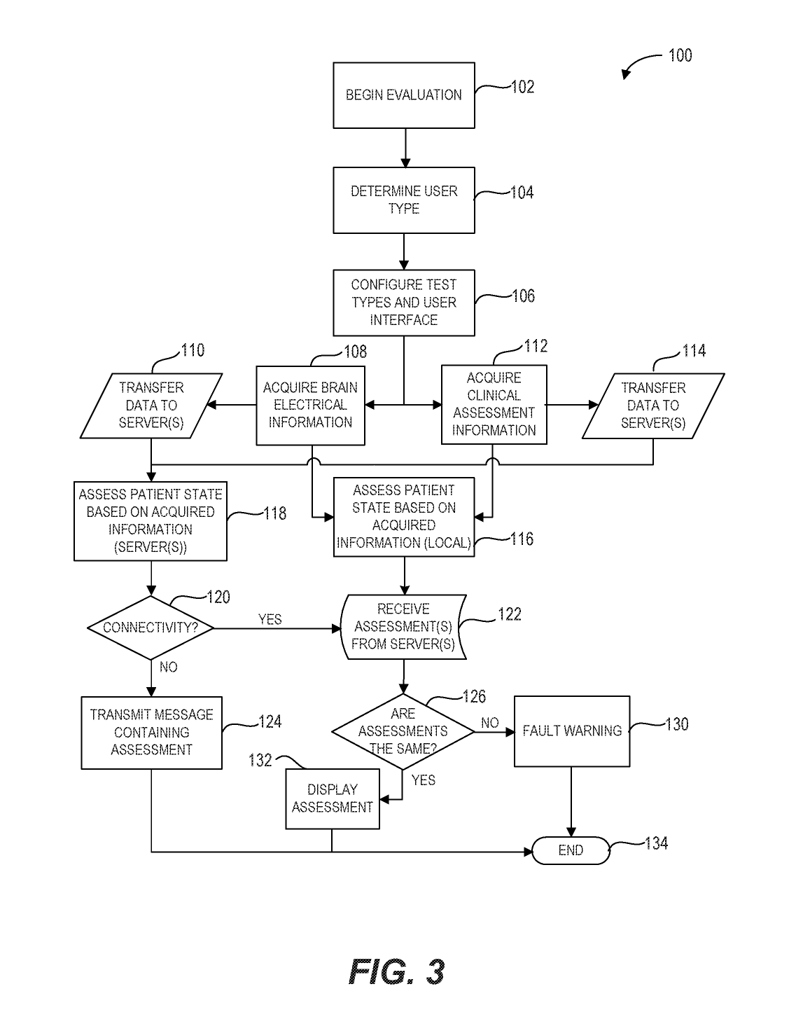

FIG. 3 depicts an illustrative process 100 for assessing a patient state using healthcare system 10. This process can begin in a designated healthcare facility, e.g., hospital or trauma unit, or in a variety of different locations such as a battlefield, in an ambulance, in a primary care setting or other physician's office, or on a sports field.

The evaluation begins at step 102, and the system 10 determines the user type of the local assessment system 20 at step 104, for instance via a login sequence or previous institutional configuration. Each user of the system 10 will have user credentials which help to identify the type of user or user group that the user belongs to. For instance, separate groups might be established for subjects, clinicians, administrators, etc., each with different privileges and each with different needs from the system 10. Each user can also be assigned a profile with configurable settings that are based on their role in the use of the system 10. Based on the user credentials, the local assessment device 20 is configured to present various test types and menus, instructions, etc. via a user interface at step 106. In an illustrative embodiment, the interface is adaptive and allows for both adaptive navigation and adaptive presentation based on the user profile. As one example, a military medic user could be identified by his or her credentials as being a user of the MACE clinical assessment tool, and the interface and system could present that test as part of the assessment run on a subject. The user interface in an exemplary embodiment could also be adaptive to that over time; explanatory information on the screen might be decreased as experience level increases, etc. A user that is identified by credentials as a subject would not likely be a user of the assessment system 20, but rather as a user of a web application 48. Users identified by credentials as institutional personnel logged into the institutional server could be identified as doctors having privileges to view patient confidential information or administrators with access to group trends and other data via terminals 36, etc.

In an exemplary user interface, where the user's credentials are associated with an emergency department (ED) clinician, the interface would present content related to ED assessment of a subject. For example, the interface could show a structural assessment, such as whether the patient was likely CT+ or CT−, and whether there was likely blood present intracranially. The interface could then present functional assessment such as EEG results outside normal ranges, results of reaction time assessments including reaction times within or without a normal range, visio-spatial recognition performance outside a normal range compared to normative results or to baseline. In other illustrative embodiments, the ED display could also include, for example, whether the patient has symptoms falling within American Academy of Neurology (AAN) guidelines such as injury above the clavicle, loss of consciousness, sensitivity to light, moderate headache. In an illustrative embodiment, the subject's score on standardized neurocognitive assessment tools can be displayed.

At step 108, brain electrical information is acquired from the local assessment device 20 and transferred to one or both of institutional server 34 and central server 58. At step 112, clinical assessment information is acquired by the user on the subject. This can happen at the same time as the electrical information is acquired at step 108 or at a different time, and can be entered on either the local assessment device 20 or on one of the various terminals 36, 46, 62. Clinical assessment information can also be obtained from sensing device 21a or additional sensing device 21b. In one illustrative embodiment, the clinical assessment information is an ocular test. In one illustrative embodiment, the clinical assessment information is collected via a tablet or handheld computer connected to the institutional or central server (34, 58), or connected to the internet 42. This information is transferred to one or both of institutional server 34 at step 114.

If connectivity is not available for data transfer steps 110, 114, the information to be transferred can be kept in a buffer or other file stored in memory transmitted when connectivity is established. Transfer to servers in steps 110, 114 can be to an institutional server, a central server, or any other server.

At step 116, the local assessment device, which can advantageously be programmed to perform the assessment locally without the need for connectivity, performed an assessment of the patient's state as described herein. In parallel with the local assessment at step 116, one or more servers runs the assessment at step 118. This parallel assessment is a safety, providing a redundant assessment in the event of the failure, error or other malfunction of local assessment device 20, or in the event of software obsolescence or error that was otherwise not detected or trapped. The redundant assessment can be run on the brain electrical information obtained at step 108, the clinical assessment information acquired at step 112, or both. At step 120, the system checks for connectivity with the local assessment device 20. If connectivity is present, then the results of the parallel assessment at step 118 is transmitted to the local assessment device 20 at step 122. If connectivity is not present, then the results can be transmitted by another means at step 124, for example by emailing or SMS or MMS messaging the results to addresses stored in the user's profile. This advantageously allows users of illustrative embodiments to receive assessment results in the event of device failure for any reason, for example if the device is dropped, shocked or broken after brain electrical signal acquisition but before the results are obtained by the user. Other transmission protocols can be used, for example satellite telephony, facsimile transmission, synthesized voice transmission over voice channels, VOIP, radio transmission, etc. Of course, if connectivity is lost, the local assessment device 20 may still be operating and may provide the results. The messaging of the redundant test analysis can, in illustrative embodiments, be turned on or off as an option in the user's profile.

At step 126, where connectivity is present at step 120, the assessment provided at step 122 is compared with the assessment calculated by the local assessment device 20 at step 126. If the two assessments are different, which can be determined by sharing consistency data between the assessment device and the redundant computing device, e.g. a server, a fault is indicated at step 130. This provides the user of the local assessment device 20 with a notification that two assessments using the same data obtained different results. This fault indication can be further refined based on device diagnostic information displayed to the user. The fault warning can be placed in the record of the assessment to flag the data or the results as potentially unreliable. If the assessments are determined to be the same, or not inconsistent according to some acceptance criteria, the device proceeds to display the assessment at step 132. The display can also include a confirmation that the redundant assessment was the same as determined at step 126. The illustrative process ends at step 134.

### Data Management

The information related to the neurological state of a subject can include a number of different data types. In addition, the central server 58 can be configured to interface with or receive information from other medical records systems such as institutional server 34. In some embodiments, the medical records systems can include, for example, hospital medical records, laboratory records, radiologic records, and/or any other data storage media. Further, it will be understood that local assessment devices 20 and terminals 36, 46 can be distributed at various locations to collect information from different personnel involved in the care of a subject or directly from a patient, for example via terminal 46. For example, the information can be derived from brain or neurological imaging studies such as CT scans, MRI, PET, angiograms, or any other suitable radiologic or imaging study that provides information related to brain structure and/or function. In addition, the information can be derived from various laboratory tests, which may be indicative of certain neurologic abnormalities. For example, suitable tests can include analyses of cerebrospinal fluid for substances indicative of infection, immunologic disorders, cerebral hemorrhage, or other neurologic processes. In addition, laboratory tests indicative of neurologic processes such as stroke, autoimmune disorders, medical or metabolic abnormalities that may affect neurologic function, presence of drugs or other substances in the blood. In some embodiments, the information can include an assessment made by a physician or other healthcare provider, including, for example, information related to physical examination or an overall assessment, differential diagnosis, or probable diagnosis based on examination and laboratory and imagining studies. The information can also include a neurologic cognitive exam based on question posed to test various neuro-cognitive abilities (e.g., memory, linguistic skills, or reasoning) or standardized assessments, for example for concussion. The information can also include ocular function testing for assessments of traumatic brain injury (TBI), including concussion, the vestibular-ocular reflex, ocular muscle balance, saccades and pursuit.

In some embodiments, one or both the institutional server 34 and the central server 58 can include one or more databases containing data related to brain electrical activity. In certain embodiments, the system can include two or more databases. One database can be used to facilitate automatic algorithm development to assist in diagnosis and treatment by collecting data related to numerous different subjects from, potentially, multiple locations. Other databases can store data related to a specific subject to allow longitudinal assessment and treatment of that subject. Illustrative systems and methods can facilitate monitoring and treatment of specific subjects over time. Accordingly, the system can be configured to store a detailed treatment record along with brain electrical activity data and other neurologic assessments. The treatment record and data related to the subject's neurologic state can be compared to other subjects within a centralized database, and based on the comparison; future treatment recommendations can be made. In addition, the response of the subject to that treatment or lack thereof can also be derived or notated in the subject's data or profile.

In various embodiments, the information stored in one or both of institutional server 34 and central server 58 related to clinical assessment information can be associated with the data related to brain electrical activity. Methods for associating the data associated with brain electrical activity with the data not including brain electrical activity are described in commonly assigned U.S. Pat. No. 8,577,451, incorporated herein by reference. In other embodiments, for example a team doctor can provide information obtained from the patient, for example CT information regarding structural injury, can update information in a player's record in order to complete information not updated on a team's institutional server from a separate institution (e.g., a hospital) because of patient confidentiality.

In order to ensure subject confidentiality while allowing data in the one or both of institutional server 34 and central server 58 memory to be updated and improved, various safe guard measures may be used. In some embodiments, the analysis system 20 is configured to transfer to the central server 58 the record identifier and not other information related to the identity of the subject. In addition, any other additional servers or assessment devices can be configured to store the record identifier and information identifying the subject in a second record, which in illustrative embodiments can be encrypted, so that a user without proper credentials accessing the data in one or both of institutional server 34 and central server 58 cannot identify a particular subject associated with a certain neurological state or diagnosis or on the basis of other confidential information. Institutional server 34, for example, may include hospital electronic record systems where a subject is being treated, and therefore, information that reveals the identity of the subject will only be available to health care providers who have a need to know such information and the matching credentials. In addition, in some embodiments, additional servers can be contained within the internet or cloud.

It may be desirable to collect information related to subjects' neurological states and to assist in providing neurological diagnoses at multiple different locations. For example, multiple healthcare facilities, military field locations or sports arenas may wish to use the neurological diagnosis capabilities provided by the database within central server 58. In addition, in order to increase the number of subjects within the stored dataset in the central server 58, multiple subject evaluation sites may be used. Accordingly, in some embodiments the system includes two or more local assessment systems 20 located at two or more different locations, and/or two or more terminals 36, 46, 62 located at two or more different locations.

As noted above, the local assessment system 20 may be configured to provide an assessment of a subject's neurological state based on brain electrical activity. However, it may be necessary to have a subject consent to the use of that information to update a database or dataset used for future subject assessment. Accordingly, in some embodiments, data collected at local assessment system 20 may be stored at the local device only, or at the local device and institutional server 34 only, and used only for initial subject assessment and treatment until a subject or other competent person is able to consent to use of the data for a desired purpose other than subject treatment.

### Other Uses of Collected Data

After data based on neurologic electrical activity and non-electrical activity has been collected, such information can be stored on central server 58 for future use in data mining and identifying correlations between various parameters within the patient data.

For example, in some embodiments, an assessment based on non-electrical clinical data could be found to correlate strongly with certain feature within the brain electrical activity, or with age or gender, etc.

As noted above, the collected information can also be used to update or train classifiers as described in commonly assigned U.S. Pat. Nos. 8,792,974, 9,198,587 and Published U.S. Patent Application No. 2014/028172.

In some embodiments, the systems and methods can be configured to store details related to specific treatments (e.g., drugs, surgeries, interventional procedures), which can be used to guide treatment planning. In addition, the systems can be configured to correlate subject responses to various treatments over time to assist in future treatment planning.

### Instrument Diagnostics

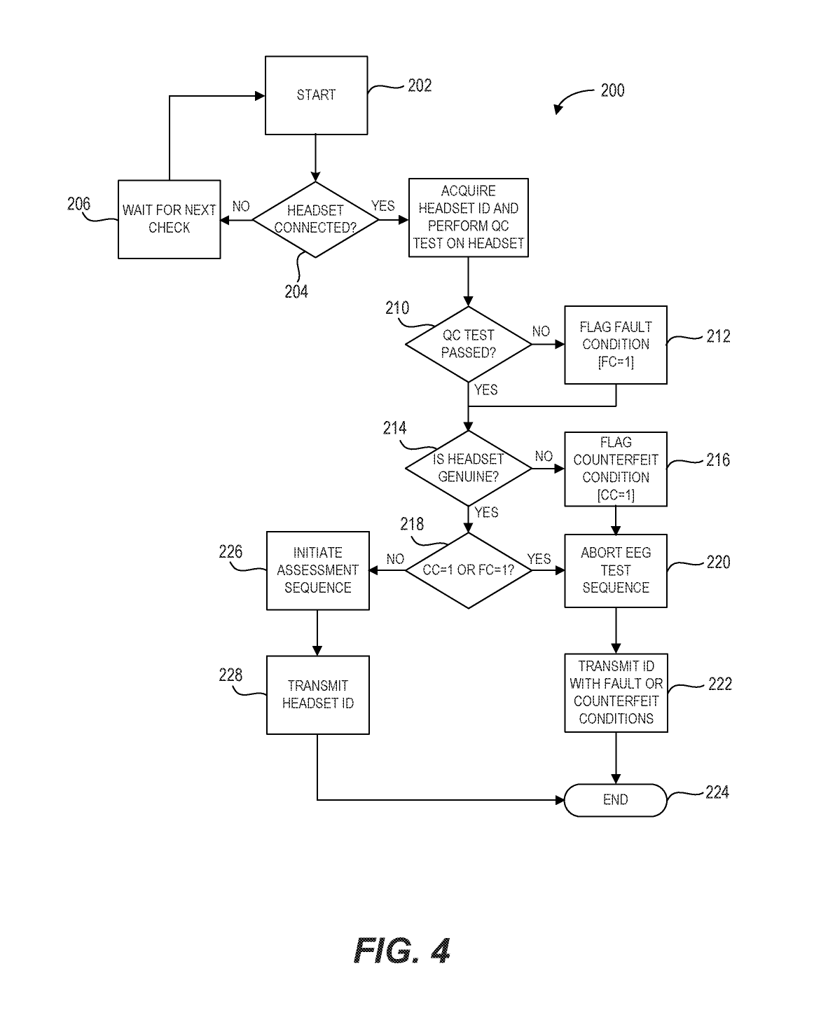

In some embodiments, network-based diagnostics can facilitate evaluation of the status of the local assessment system 20. As depicted in FIG. 4, an exemplary process 200 for performing a diagnostic of a headset attached to an assessment system 20 is described. The process begins at step 202. And step 204 the device checks to see if a headset has been connected to the assessment system 20. The assessment device can be used without a headset to perform clinical assessments, access stored or remote accessed results, to display information, etc. If a headset is not detected at step 204, the device can enter a pause loop or otherwise schedule a check for a headset at some point in the future at step 206. Optionally, the headset connector can contain a means for creating an interrupt or other signal indicating a headset is present. In such an arrangement, the wait of step 206 could be very short, or nearly instantaneous depending on the hardware implementation. At step 208, once a headset has been detected at step 206, a headset ID is acquired from the headset, which has an electronic storage device containing the ID. A quality control test can also be performed, for example an impedance check of the headset to ensure the electrodes and signal lines are within operational limits. At step 210, a pass or fail decision is taken. If the headset fails the QC testing, a flag is set to indicate a fault condition. The process continues to step 214, where a check is run on the headset ID to determine authenticity. If the headset is determined to not be authentic, a flag is set at step 216 to indicate the headset is counterfeit. The local assessment device 20 can contain the data necessary for confirming authenticity. Alternatively, the authenticity test is determined on a server, and the result transmitted back to the local device. In one embodiment, the authenticity check can include an encrypted and authenticated challenge and response between the assessment device 20 and the electronic storage device on the headset respectively. The electronic storage device on the headset must be securely programmed with the appropriate cryptographic key(s) at the time of manufacture. If connectivity is not available, then the process can buffer or store the counterfeit test request and/or result in memory for transmission when connectivity is available. At step 218, the process checks if a flag has been set, for either genuineness at step 214 or QC conformance at step 210, and if a flag has been set, the test is aborted at step 220 and a message displayed to the user indicating termination of the test. At step 222, the existence of a fault condition or counterfeit condition is transmitted via any available means. Advantageously, the data allows tracking of the field operation of local assessment systems 20, as well as the precise location of counterfeit headsets. If the counterfeit status cannot be determined at step 214, the assessment would be allowed to proceed, with an appropriate warning to the user that headset genuineness could not be confirmed. The genuineness test could be performed at a later time, and appropriate notifications provided to the user, institution or central server in due course, as soon as possible.

If no fault or counterfeit conditions are detected at step 218, the subject assessment sequence is initiated at step 226, and the device is capable of performing an assessment of a subject's electrical brain activity. At step 228, the headset ID is transmitted, via any available means, to eventually provide the manufacturer with a record of a genuine headset having been used. Advantageously, this allows a database of used device IDs to be maintained, so that counterfeits are not able to reuse a previously used headset ID to bypass the authenticity check. This exemplary subprocess ends at step 224.

### Management of Subject Information

The management of a subject's brain health after over time can be implemented as part of the assessment system as described in commonly assigned U.S. Pat. No. 8,579,812, which is incorporated herein in its entirety by reference. In illustrative embodiments, a user of the local assessment device 20 could ask the subject if they would like information sent to them, for example by email, on brain health, traumatic brain injury, concussion, etc. The user interface could be configured to offer options as to the type of information that would be relevant to the user's profile and ultimately the assessment of the subject. For example, information available to a military medic for subjects experiencing the concussive effects of ordnance explosion might vary from information available to a youth sports coach for youth athletes experiencing a head blow during a game. In an illustrative embodiment, the user is an urgent care clinic practitioner, where information available might comprise:

i. Overview of TBI and Concussion;

ii. Signs and Symptoms and other indicators;

iii. Venue-specific or location-specific information, such as information on resources or contacts available;

iv. Caregiver information;

v. Age-specific information for the subject;

vi. Listing of other public websites for information

vii. A link to the BrainScope web application 48 along with data that will allow the central sever 58 to authenticate the user add allow access.

In illustrative embodiments, the local assessment device 20 can contain GPS sensors to ascertain geographical position, and calculate context or resources based on that calculation. For example, placement at a sports venue might indicate an athletic event, a conflict zone might indicate a battlefield location, a hospital location might indicate a clinical setting, etc. Location information can be supplemented by other data for locating services, facilities or other users nearby, and linked services can provide directions using navigational aids.

In exemplary embodiments, the local assessment device 20 could present a sign-off or acceptance screen for the subject to indicate endorsement of the choice and authorization to send information. The information can be presented via text, HTML, an app for mobile device, or links to web sites or microsites containing useful information. Information could be sent via email, SMS or MMS message, or other messaging protocol to the subject, the subject's caregivers, or other recipients.

The results of the assessments, or other information contained in the patient's profile, could be used to populate the information presented to the subject in the message or the microsite, for example. By way of non-limiting further example, the information could include age-specific information, injury-specific information, and special information for those where a current injury is a repeat injury, etc.

### Neurologic Panel

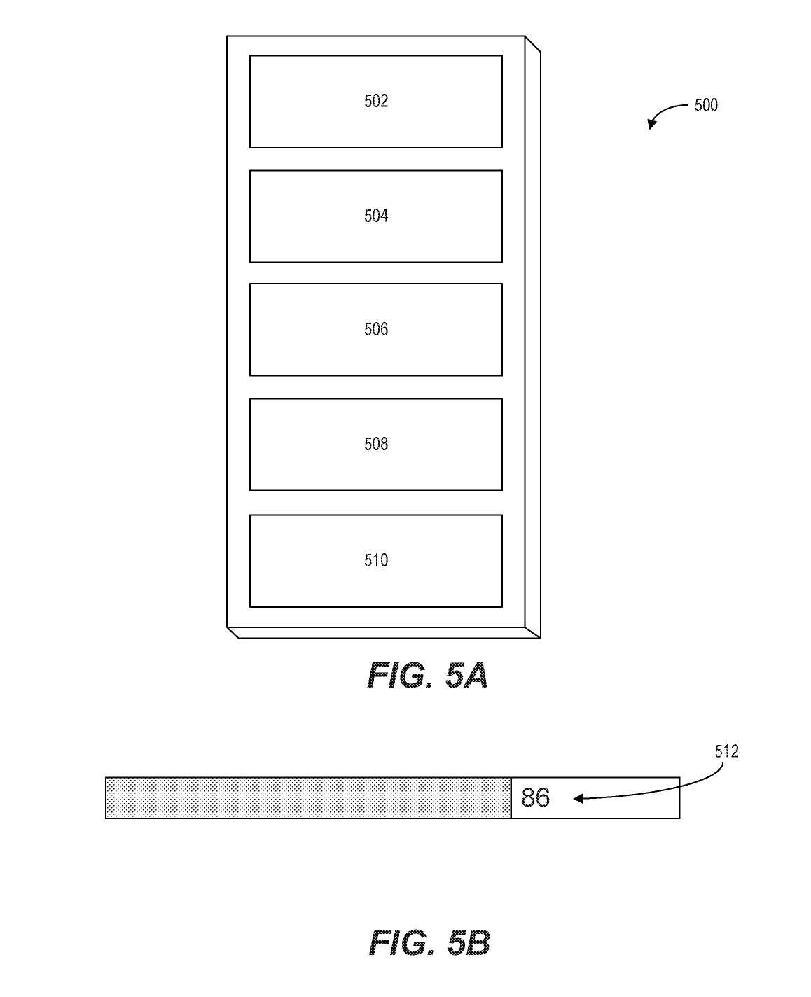

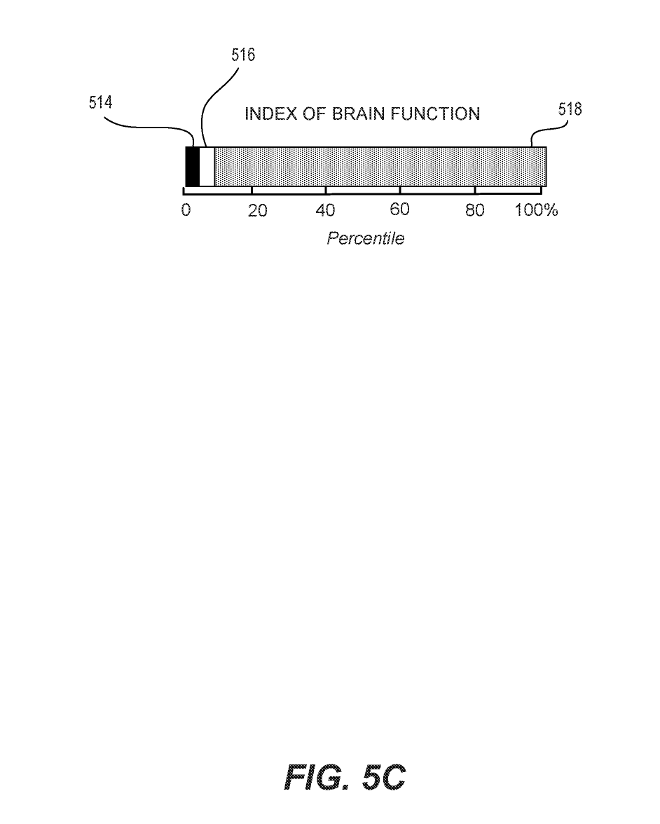

Turning now to FIG. 5A, there is depicted an illustrative user interface output of the various aspects of the neurological assessments performed by or obtained by the healthcare system 10. As described above, various assessment modalities can be performed by the local assessment device 20. As well, information of a subject's neurologic state can be stored on servers and transmitted to the local assessment device 20. Any or all of these assessments can be displayed on interface 500 at exemplary locations 502, 504, 506, 508 and 510 to form a results panel. Of course the number of locations may vary by the number of assessments. The results panel may not only display the overall neurologic state of a subject, but also a comprehensive summary of the results of any brain electrical activity assessment and neurocognitive and clinical symptoms assessments performed on the subject.

FIG. 5B depicts an exemplary representation of a graphical component which can form a part of the assessment displays 502, 504, 506, 508, and 510. The graphical component can show an index value 512 to indicate the patient's assessment as compared to a norm, or in the case of more statistically involved calculations, can reflect the probability of correctness of a neurological assessment, as discussed in U.S. Pat. No. 8,364,254, which is incorporated by reference in its entirety.

FIG. 5C depicts an exemplary representation of an index of brain function or normality indicator, which can form a part of the assessment displays 502, 504, 506, 508, and 510. The brain function index may graphically depict three or more ranges on a percentile scale, as illustrated in FIG. 5C. In exemplary embodiments, the brain function index may depict the following three ranges: 1) within normal range 514 (e.g., >10^{th} percentile), 2) below normal range 516 (e.g., ≦10^{th} percentile and >2^{nd} percentile), and 3) clearly below normal range 518 (e.g., 2^{nd} percentile). In such an embodiment, if a subject's score falls in the 84^{th} percentile, the subject will clearly fall within the normal range, thus indicating that the subject is exhibiting normal brain state. If the subject's score is in the 4^{th} percentile, the subject will be considered below the normal range.

Advantageously, presenting all neurologic measures on a panel provides a user with an overall view of the subject's neurological state.

### Conclusion

The systems and methods disclosed herein are not inherently related to any particular computer, electronic control unit, or other apparatus and may be implemented by a suitable combination of hardware, software, and/or firmware. Software implementations may include one or more computer programs comprising executable code/instructions that, when executed by a processor, may cause the processor to perform a method defined at least in part by the executable instructions. The computer program can be written in any form of programming language, including compiled or interpreted languages, and can be deployed in any form, including as a standalone program or as a module, component, subroutine, or other unit suitable for use in a computing environment. Further, a computer program can be deployed to be executed on one computer or on multiple computers at one site or distributed across multiple sites and interconnected by a communication network. Software embodiments may be implemented as a computer program product that comprises a non-transitory storage medium configured to store computer programs and instructions, that, when executed by a processor, are configured to cause the processor to perform a method according to the instructions. In certain embodiments, the non-transitory storage medium may take any form capable of storing processor-readable instructions on a non-transitory storage medium. A non-transitory storage medium may be embodied by a compact disk, digital-video disk, hard disk drive, a magnetic tape, a magnetic disk, flash memory, integrated circuits, or any other non-transitory digital processing apparatus or memory device.

Although the foregoing has been described in some detail for purposes of clarity, it will be apparent that certain changes and modifications may be made without departing from the principles thereof. It will be appreciated that these systems and methods are novel, as are many of the components, systems, and methods employed therein. It should be noted that there are many alternative ways of implementing both the processes and apparatuses described herein. Accordingly, the present embodiments are to be considered as illustrative and not restrictive, and the inventive body of work is not to be limited to the details given herein, but may be modified within the scope and equivalents of the appended claims.

Other embodiments will be apparent to those skilled in the art from consideration of the specification and practice of the devices and methods disclosed herein. It is intended that the specification and examples be considered as exemplary only, with a true scope being indicated by the following claims.

What is claimed is:

1. An apparatus for monitoring for evaluating a neurologic state of a subject by a user, comprising:
one or more electrodes disposable on the subject and configured to detect brain electrical signals of the subject;
an assessment device having a processor and a memory, the assessment device configured to receive the brain electrical signals and the processor configured to perform a first assessment of the brain electrical signals;
a computing device, separate from the assessment device, having a processor and a memory; and
a first communications channel between the assessment device and the computing device, wherein brain electrical signals can be communicated from the assessment device to the computing device when the first communications channel is open;
wherein the computing device processor performs a second assessment of the brain electrical signals.

2. The apparatus of claim 1, wherein:
the assessment device is configured to transmit data representative of the results of the first assessment to the computing device;
the computing device compares the first assessment and the second assessment with the processor; and
the computing device records to memory consistency data representing whether the results of the first assessment and second assessment are consistent.

3. The apparatus of claim 2, wherein the computing device is configured to transfer consistency data to the assessment device indicative of the comparison of the first assessment and the second assessment.

4. The apparatus of claim 3, wherein the first assessment and the second assessment employ substantially the same assessment redundantly.

5. The apparatus of claim 3, wherein the consistency data is transferred from the computing device to the assessment device via the first communications channel.

6. The apparatus of claim 3, wherein the consistency data is transferred from the computing device to the user via a second communications channel.

7. The apparatus of claim 6, wherein the computing device is configured to ascertain whether the first communications channel is open.

8. The apparatus of claim 2, wherein:
the assessment device is located a first location;
the computing device is located a second location; and
the first location is different from the first location.

9. The apparatus of claim 1, wherein the assessment device is further configured to receive neurocognitive assessments and/or clinical symptoms assessments.

10. The apparatus of claim 9, wherein the neurocognitive assessments and/or clinical symptoms assessments are input to the assessment device by the user.

11. The apparatus of claim 9, wherein the neurocognitive assessments and/or clinical symptoms assessments are transferred to the assessment device from the computing device on the first communications channel.

12. The apparatus of claim 1, wherein the assessment device is further configured to receive longitudinal information of the subject's neurological state.

13. The apparatus of claim 1, wherein the computing device is further configured to provide a treatment recommendation based data stored in the database and a classification of the neurologic state of the subject.

14. The apparatus of claim 13, wherein the computing device is configured to transmit the treatment recommendation to the assessment device on a first communications channel.

15. The apparatus of claim 13, wherein the computing device is configured to transmit the treatment recommendation to the user on a second communications channel.

16. The apparatus of claim 1, wherein:
the assessment device is configured to extract features from the subjects brain electrical activity, neurocognitive assessments and/or clinical symptoms assessment;
the assessment device is configured to determine whether values of the extracted feature fall within or outside their normal ranges, wherein the normal ranges are defined by a reference database comprising data collected from a control population group exhibiting normal brain function.

17. The apparatus of claim 16, wherein the assessment device is configured to calculate a summary index score using normal and abnormal features; and
the assessment device is configured to generate a brain function index using the index score, wherein the brain function index expresses the neurologic state of the subject as a percentile relative to normal brain function exhibit by the control population group.

18. The apparatus of claim 1, wherein:
the computing device is configured to extract features from the subjects brain electrical activity, neurocognitive assessments and/or clinical symptoms assessment;
the computing device is configured to determine whether values of the extracted feature fall within or outside their normal ranges, wherein the normal ranges are defined by a reference database comprising data collected from a control population group exhibiting normal brain function.

19. The apparatus of claim 18, wherein the computing device is configured to calculate a summary index score using abnormal features; and
the computing device is configured to generate a brain function index using the summary index score, wherein the brain function index expresses the neurologic state of the subject as a percentile relative to normal brain function exhibit by the control population group.

20. The apparatus of claim 18, wherein the reference database comprises one or more of brain electrical activity data, neurocognitive assessments data and clinical symptoms data.
